(19) United States
(12) Plant Patent Application Publication
(10) Pub. No.: US 2012/0210479 P1
(43) Pub. Date: Aug. 16, 2012

(54) Crapemyrtle plant named 'GAMAD IX'

(50) ;

(75) Inventor: Michael A. Dirr, Bogart, GA (US)

(73) Assignee: University of Georgia Research Foundation, Inc., Athens, GA (US)

(21) Appl. No.: 12/932,043

(22) Filed: Feb. 16, 2011

(51) Int. Cl. A01H 5/00

(52) U.S. Cl. PLT/252

(57) ABSTRACT

A new and distinct cultivar of crapemyrtle, ‘GAMAD IX’, is provided. ‘GAMAD IX’ is a Lagerstroemia indica×Lagerstroemia fauriei hybrid, which is characterized by intermediate growth habit, mildew resistance, and earlier flowering with abundant purple flowers.

### LATIN NAME OF THE GENUS AND SPECIES OF THE PLANT CLAIMED

‘GAMAD IX’ is a crapemyrtle plant that is a Lagerstroemia hybrid.

### VARIETY DENOMINATION

The new crapemyrtle plant claimed is of the variety denominated ‘GAMAD IX’.

### BACKGROUND OF THE INVENTION

The present invention relates to the discovery of a new and distinct cultivar of the ornamental flowering shrub Lagerstroemia indica×Lagerstroemia fauriei, commonly known as crapemyrtle, and hereafter referred to by the varietal denomination ‘GAMAD IX’, as herein described and illustrated.

The new crapemyrtle originated from open-pollinated seed of an unpatented seedling. ‘GAMAD IX’ was derived from plants grown at Dearing, Ga. from these seeds. The seedlings were planted in containers and selections were made for plants based on the following criteria: 1) intermediate growth habit; 2) mildew resistance; 3) early flowering; and 4) flower color and quality. ‘GAMAD IX’ was selected in 2004.

Asexual reproduction by traditional vegetative cuttings since 2004 at Dearing, Ga. and in Athens, Ga. has shown that the distinguishing characteristics of this new crapemyrtle variety ‘GAMAD IX’ are stable and reproduced true-to-type in successive generations.

### SUMMARY OF THE INVENTION

The new crapemyrtle plant variety ‘GAMAD IX’ has not been observed under all possible environmental conditions. The phenotype may vary somewhat with variations in environment and cultural practices such as temperature and light intensity without, however, any variance in genotype.

The following traits have been repeatedly observed at Dearing, Ga. and Athens, Ga., and are determined to be unique characteristics of the new crapemyrtle plant variety ‘GAMAD IX’:

1. Intermediate size

2. Mildew resistance

3. Earlier flowering in mid-to late June (USDA Zone 7)

4. Abundant purple flowers

There were no intermediate purple flowering varieties of crapemyrtle available for comparison. ‘GAMAD IX’ is similar in color to ‘Catawba’ (unpatented), but has a much smaller overall habit and is earlier flowering with abundant purple flowers.

### BRIEF DESCRIPTION OF THE DRAWINGS

The accompanying photographic illustrations show typical specimens in full bloom of the new variety ‘GAMAD IX’. The colors are as nearly true as is reasonably possible in a color representation of this type.

FIG. 1 is a photograph of the new variety ‘GAMAD IX’.

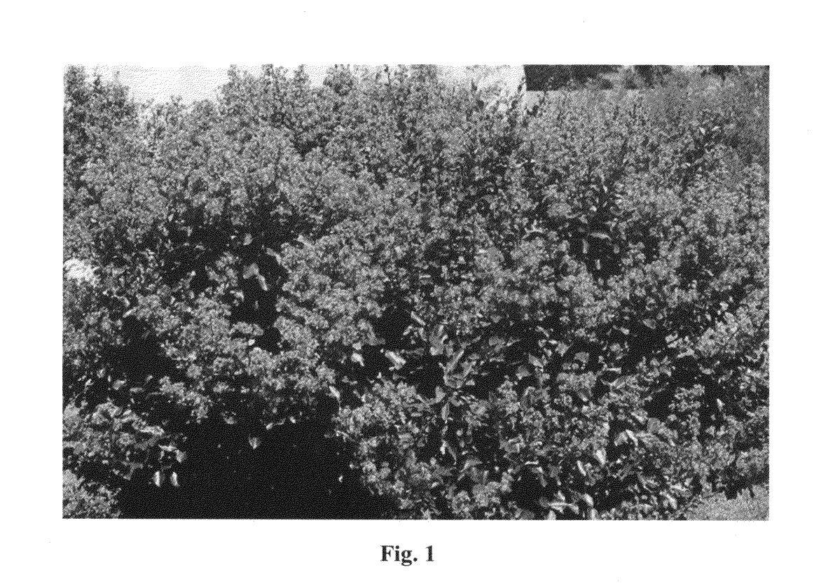

FIG. 2 is a photograph more closely illustrating the flowers of the new variety ‘GAMAD IX’.

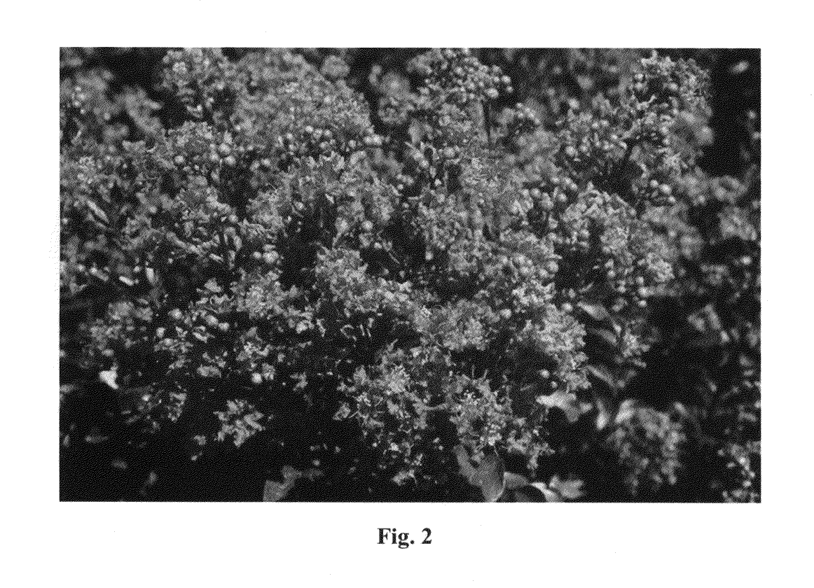

### BOTANICAL DESCRIPTION

Throughout this specification, color names beginning with a small letter signify that the name of that color, as used in common speech, is aptly descriptive. Color names beginning with a capital letter designate values based upon the R.H.S. Colour Chart, 5^{th} edition published by The Royal Horticultural Society, London, England.

The following is a detailed description of the botanical and horticultural characteristics of the new variety ‘GAMAD IX’. Where dimensions, sizes, colors, and other characteristics are given, it is to be understood that such characteristics are approximations and averages set forth as accurately as practicable. The descriptions reported herein are from specimen plants grown at Athens, Ga. Data were obtained in summer 2010 on plants that were 4 years old.
Plant:
   Habit.—Intermediate, rounded to broad-rounded.
   Size.—Height: 140 cm. Width: 180 cm.
   Texture.—Medium.
Stems:
   Color.—Greyed-brown RHS 199C in first year changing to Greyed-brown RHS 200D in the second year.
   Diameter.—2 mm.
   Pubescence.—Observed along edges.
   Exfoliation.—Strips.
   Shape.—Round.
   Pith.—Type: Solid. Diameter: 1 mm. Color: Green RHS 137A.
   Odor (of bruised stem).—None.
   Lenticels.—None.
   Internode length.—4.5 mm to 21 mm.
Vegatative buds:
   Arrangement.—Sub-opposite to alternate.
   Type.—Imbricate, ovoid to rounded.
   Size.—Length: 3 mm. Width: 1 mm.
   Scale number.—3.
   Scale color.—Greyed-brown RHS 199D.
   Position/disposition.—30 degrees to stem.
   Number at node.—1.
   Pubescence.—None.
   Shape.—Ovoid to rounded.
Leaf scar:
   Shape.—Raised half ellipse.
   Vascular bundle traces.—None observed.
   Pubescence.—None.
   Position of bud.—Directly on top.
   Color differentiation.—Green RHS 137A.
   Size.—Height: 1 mm. Width: 2 mm.
Trunk or large stems:
   Color.—Grey-brown RHS 199C.
   Size of stem on which exfoliation begins.—14 mm, with some peeling occurring.
   Diameter.—14 to 19 mm.
   Texture.—Slightly exfoliating.
Leaf:
   Color at emergence.—Upper: Yellow-green RHS 148A. Lower: Yellow-green RHS 148B.
   Color during summer season.—Upper: Dark-green RHS 136A. Lower: Green RHS 138A.
   Mature size.—Length: 50 mm (on average). Width: 32 mm (on average).
   Apex.—Acuminate.
   Base.—Acute.
   Margin.—Entire, undulating.
   Shape.—Obovate.
   Vein color.—Yellow-green RHS 146D.
   Pubescence.—Finely hairy on entire upper leaf surface, only observed along veins and midrib on lower surface.
   Arrangement on stem.—Sub-opposite to alternate.
   Venation.—Pinnate.
   Texture.—Leathery. Thickness: 0.5 mm thick. Degree of waxiness of surfaces: Moderately waxy.
Petiole:
   Length.—2 mm.
   Shape.—Mostly rounded to semi-oval.
   Color.—Grey-purple RHS 187B on top and Yellow-green RHS 146D underneath.
   Pubescence.—Finely hairy.
   Diameter.—1 mm.
Flower buds:
   Size.—Length: 7 mm. Width: 5.5 mm.
   Color: Greyed-red RHS 178A.
   Shape.—Oval-rounded.
   Pubescence.—None.
   Time of full maturity.—Mid to late June.
   Time range for showiness.—Approximately 9 to 11 weeks for buds and flowers.
Flower:
Inflorescences:
   Type.—Paniculate.
   Size.—Length: 9.25 cm. Width: 7.5 cm.
   Color at emergence.—Red-purple RHS 59A.
   Color at full bloom.—Red-purple RHS 59A.
   Color at fading.—Purple RHS N79A.
   Peduncle.—Color: Greyed-purple RHS 183C. Pubescence: Finely hairy along edges.
Petals:
   Number.—6.
   Size.—Length: 21 mm. Width: 15 mm.
   Shape.—Fan-shaped.
   Apex.—Ruffled, rounded.
   Base.—Sagittate.
   Margin.—Ruffled.
   Pubescence.—None.
   Texture.—Smooth.
   Color at peak of bloom.—Upper surface: Red-purple RHS 59A. Lower surface: Red-purple RHS 59A.
   Pedicels.—Color: Greyed-purple RHS 185A. Pubescence: None. Length: 5.5 mm.
Male reproductive structures:
   Number.—6 (fertile); 20 to 30 (sterile).
   Pollen color.—Yellow-orange RHS 14A.
   Pubescence.—None.
Anther:
   Size.—(Both fertile and sterile). Length: 1.5 mm. Width: 1 mm.
   Color.—Yellow-green RHS 144A (fertile); Yellow-orange RHS 14A (sterile).
Filament:
   Size.—8 mm to 12 mm (fertile); 8 mm to 10 mm (sterile).
   Color.—Red-purple RHS 60B (fertile); Red-purple RHS 69B (sterile).
Female reproductive structures:
Pistil:
   Size.—Length: 16 mm. Width: 0.5 mm.
   Position.—Superior.
   Pubescence.—None.
Stigma:
   Shape.—Rounded.
   Color.—Yellow-green RHS 148A.
   Pubescence.—None.
Style:
   Length.—16 mm.
   Shape.—Linear.
   Color.—Red RHS 46A.
   Pubescence.—None.
Ovary:
   Shape.—Oval to round.
   Number.—One per flower.
   Pubescence.—None.
   Size (diameter).—2 mm.
   Color.—Yellow RHS 3C.
Fruit:
   Type.—Dehiscent, six-valved capsule.
   Size.—Length: 9 mm. Width: 6 mm to 7 mm.
   Color during ripening.—Early: Greyed-orange RHS 165B at tip; Yellow-green RHS 146C at base. Mid: Yellow-green RHS 146B. Late: Brown RHS 200B (before splitting); Greyed-orange RHS 165A (after seed dispersal).
   Shape.—Broad-ellipsoidal.
   Number per infructescence.—80 to 90.
   Pubescence.—None.
   Number of carpels.—6.
   Persistence (effective period).—3 months holding into winter.
Seed:
   Shape.—Oval-rounded.
   Size.—Length: 5 mm. Width: 2 mm.
   Color.—Brown RHS 200B (seed); about Greyed-orange RHS 164C (wing).
   Number per fruit.—15 to 30.
   Pubescence.—None.

What is claimed is:

1. A new and distinct variety of crapemyrtle plant named ‘GAMAD IX’, substantially as illustrated and described herein.
